(19) United States
(12) Patent Application Publication
(10) Pub. No.: US 2015/0144746 A1
(43) Pub. Date: May 28, 2015

(54) Medical Line Organizer

(71) Applicant: Benjamin Brian Stewart, Lewisburg, TN (US)

(72) Inventor: Benjamin Brian Stewart, Lewisburg, TN (US)

(21) Appl. No.: 14/543,873

(22) Filed: Nov. 17, 2014

Related U.S. Application Data
(60) Provisional application No. 61/910,098, filed on Nov. 28, 2013.

(51) Int. Cl. A61M 39/00; F16L 3/10; F16L 3/223

(52) U.S. Cl. CPC A61M 39/00; F16L 3/2235; F16L 3/1091; A61M 2209/082
USPC 248/67.5; 248/49; 29/428

(57) ABSTRACT

A device and associated method for organizing lines comprising a base structure forming a channel with two sides, the two sides having a plurality of tabs and slots between the tabs for locating and supporting medical lines, the device including a cover structure for captivating the lines and further including a resilient structure for providing friction strain relief for the lines to relieve the full weight of the lines on the patient. The device accommodates a wide size range of different lines and allows break away tabs to further increase the size range accommodated. The structure may be cut or snapped to desired length at the point of use. The device and installed lines may be easily attached and detached from the bed allowing maximum patient mobility. The cover may be easily labeled to indicate each line. Variations may be adapted for sterilization or made for one-time use.

### RELATED APPLICATIONS

The present application claims the benefit under 35 USC 119(e) of prior provisional application 61/910,098, titled: “ IV and Tube Organizer”, filed 28 Nov. 2013 by Benjamin Stewart, which is incorporated herein by reference in its entirety.

### TECHNICAL FIELD

The present disclosure relates generally to the field of organizing wires, tubes, and other lines, and more particularly to the field of organizing and supporting medical lines.

### BACKGROUND

Hospitals, clinics, surgery centers, and other medical providers routinely attach various lines to patients, often numerous lines for various purposes. These lines may include intravenous (IV) lines for hydration, analysis, medicine delivery, EKG lines and pulse oximeter lines for monitoring, oxygen lines for breathing oxygen, other sensors and special purpose lines for dialysis or other functions. These lines tend to drape to the floor, potentially picking up contamination, and stretch between the associated machine and the patient, placing considerable weight load tugging on the patient. These lines may come in a wide array of sizes and makeup from small wires and cables to tubes of multiple sizes. Attendants may wrap and tape the lines to secure them and ease the load on the patient. In the process, the numerous lines may become entangled and mixed. The process is further complicated when attendants change shifts. The new attendant must figure out which line is which for changing medications or updating treatments. Then, the lines must be untangled to change machines. If the patient needs to move to the bathroom or walk down the hall, the lines must be untangled from the bed support to move with the patient. Thus, there is a need for organizing patient lines to improve the efficiency, reliability, and sanitation of the patient care process.

### BRIEF SUMMARY OF THE DISCLOSURE

Briefly, the present disclosure pertains to a device and associated method for organizing lines comprising a base structure forming a channel with two sides, the two sides having a plurality of tabs and slots between the tabs for locating and supporting medical lines, the device including a cover structure for captivating the lines and further including a resilient structure for providing friction strain relief for the lines to relieve the full weight of the lines on the patient. The device accommodates a wide size range of different lines through the same device and allows break away tabs to further increase the size range accommodated. The structure may be cut or snapped to desired length at the point of use. The device and installed lines may be easily attached and detached from the bed allowing maximum patient mobility. The cover may be easily labeled to indicate each line. Variations may be adapted for sterilization or made for one-time use.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 illustrates an isometric view of an exemplary organizer in accordance with the present disclosure.

FIG. 2 and FIG. 3 illustrate an end view and a side view respectively of the exemplary organizer concept similar to FIG. 1, but having a longer length.

FIG. 4a shows a cross section of the exemplary organizer of FIG. 1 taken through the width of the organizer at a slot position.

FIG. 4b illustrates an exemplary alternative resilient structure.

FIG. 5 shows a top view of the exemplary organizer base section loaded with various tubes and wires.

FIG. 6a through FIG. 6c show various views of the exemplary cover concept of the organizer of FIG. 1.

FIG. 7 illustrates an exemplary holder for the organizer for attaching to a support such as, for example, a bed rail.

FIG. 8a and FIG. 8b illustrate exemplary holders for supporting the organizer and attaching the organizer to a bed rail or other support.

FIG. 9 illustrates a side view of an alternative exemplary cover key comprising a post in the base extending through a hole in the cover.

### DETAILED DESCRIPTION

This disclosure is directed to an organizer device and associated method for assistance in organizing various tubes and lines that are typically encountered in a hospital environment. The organizer comprises a base having tabs and slots for separating and organizing the lines and a cover for securing the lines within the slots and providing friction strain relief. The cover also provides a convenient surface for labeling the lines. Within this disclosures, the term lines typically includes tubes, wires, cables, strings, singly or bundled. These lines typically include, but are not limited to intravenous (IV) tubes, oxygen tubes, EKG wires, pulse oximeter wires, dialysis tubes and other sensors. The organizer fixes the lines in place, preventing tangling and movement of the lines. The organizer also provides strain relief to prevent the full weight of the lines from being carried by the patient and keeps the lines from dragging on the floor.

Further, the organizer easily accommodates lines and particularly tubes of widely varying diameters, allowing control of EKG wires along with IV and dialysis tubes in the same device. The entire organizer loaded with lines is easily attached and detached to a bed or other support for easy mobility of the patient without removing lines from the organizer.

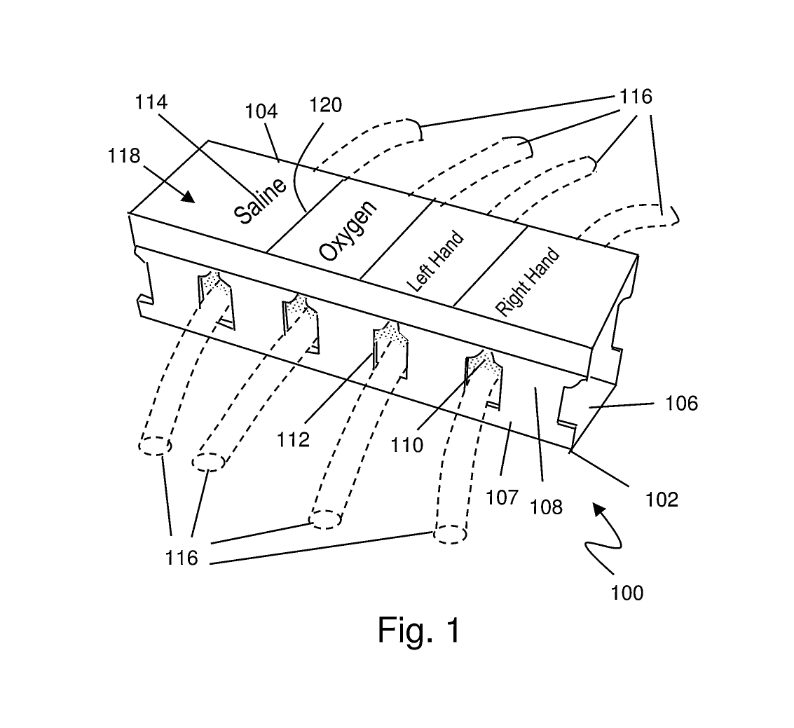

FIG. 1 illustrates an isometric view of an exemplary organizer in accordance with the present disclosure. Referring to FIG. 1, the organizer 100 comprises a base structure 102 (also referred to as a base) and a cover assembly 104 (also referred to as a cover). The base structure 102 comprises a bottom 106 and two sides 107 forming a channel structure. Each side comprises a side wall and a plurality of tabs 108. The tabs are separated at the top and sides of each tab. The space between two tabs forms a slot 112 for passing a medical line 116 through the organizer 100. The line 116 passes through a slot 112 on one side and through a corresponding slot on the other side directly across from the first slot. One or more tabs may be configured with a catch or latch structure (typically comprising a notch) to releasably engage and hold the cover assembly 104. The cover assembly comprises a channel shaped structure 104 having a complementary catch structure on each side to engage the catch structure on the tabs and releasably snap onto the base structure 102. The cover assembly also comprises a resilient structure 110 to press onto the medical lines 116 to produce a friction holding force to keep the lines in place longitudinally. The base tabs 108 and base sidewall 102 retain the lines laterally and vertically. The bottom of each slot includes an edge (base sidewall) that further provides friction for additional longitudinal holding of each line. The top of the cover 104 provides a flat surface 118 that may be used for placing labels 114 for each line, either by direct writing on the cover 104 or by placing a stick-on label, as desired. FIG. 1 also shows markings 120 on the cover half way between the lines below to help center any labels placed on the cover by caregivers to reduce confusion by potentially off center labels.

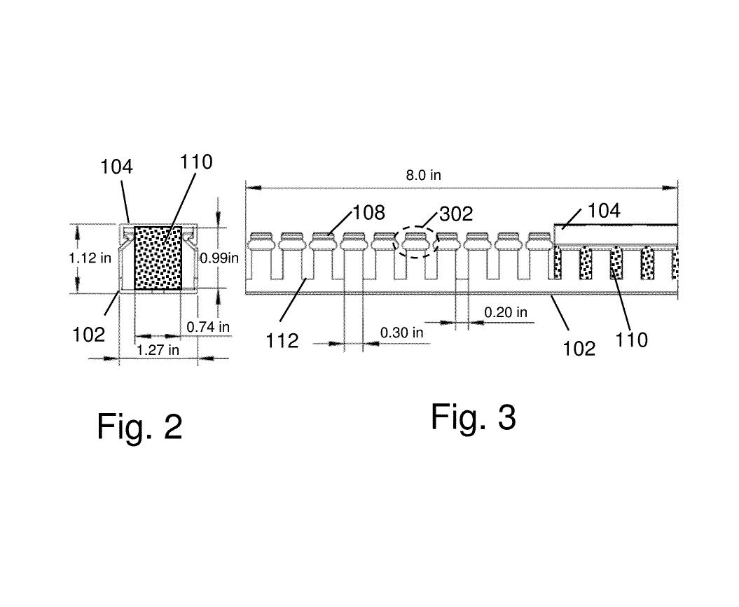

FIG. 2 and FIG. 3 illustrate an end view and a side view respectively of the exemplary organizer concept of FIG. 1, but FIG. 3 shows a longer length than FIG. 1. FIG. 2 shows an end view showing the base and cover assembly. FIG. 2 shows greater detail of the catch structures on the base tabs and cover that allow the cover to snap onto the tabs 108 of the base. FIG. 2 also shows a foam variation for the resilient structure 110 that holds the lines captive within the slots 112. Exemplary dimensions are given. Other dimensions may be chosen as desired.

FIG. 3 shows a side view of the base and cover. The cover 104 is shown as a partial length cut away to reveal detail of the base tabs 108. Exemplary dimensions are shown. The dimensions may be varied as desired. The length of the organizer may be any desired length. The linear dimension may be scaled as desired and the number of tabs may be any desired number. In FIG. 3, the tabs are shown as all the same width and slots of all the same width. Alternatively, the tabs and/or slots may be of multiple widths to accommodate different tubing or line sizes.

FIG. 3, reference 302 shows a side view of the top of the base tab. Toward the top the tab widens to constrain the lines while loading each notch. At the top of the tab 302, there is a latch structure, e.g. beveled notch to hold the cover. See FIG. 2 for the end view. Further, all tabs are shown with the latch structure 302. Alternatively some of the tabs may have the latch structure and some may omit the latch structure. Sufficient latch structures should be provided to hold the cover. In a further alternative, the pattern of latch structures may be configured to prevent attachment of the cover in reversed direction.

The linear repetitive tab structure of the base combined with the lateral snap on feature of the cover allows the organizer to be constructed in long lengths and then later cut to size as desired. The cutting to size may be performed at the point of use in the hospital, if desired. In one variation, perforations or scoring may be provided between each slot and at corresponding lengths on the cover to allow break apart separation to customize the length without using any special tools. Since each tab has its own snap feature, any length will come with sufficient snap structure to retain the corresponding cover length.

In one variation, the tabs 108 may be individually broken off or bent to allow joining two adjacent slots to accommodate larger tubing or bundles of tubing The tabs may be scored or perforated at the base to facilitate the break-off operation.

In one variation, the base and cover may be similar to PANDUIT® F1X1WH6 wire duct and cover C1WH6, modified with the addition of a resilient structure to provide strain relief for the tubing.

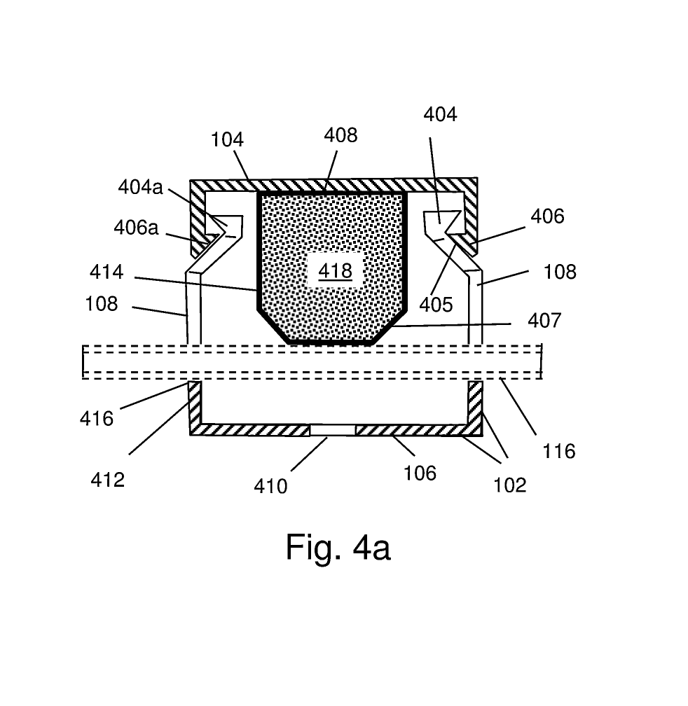

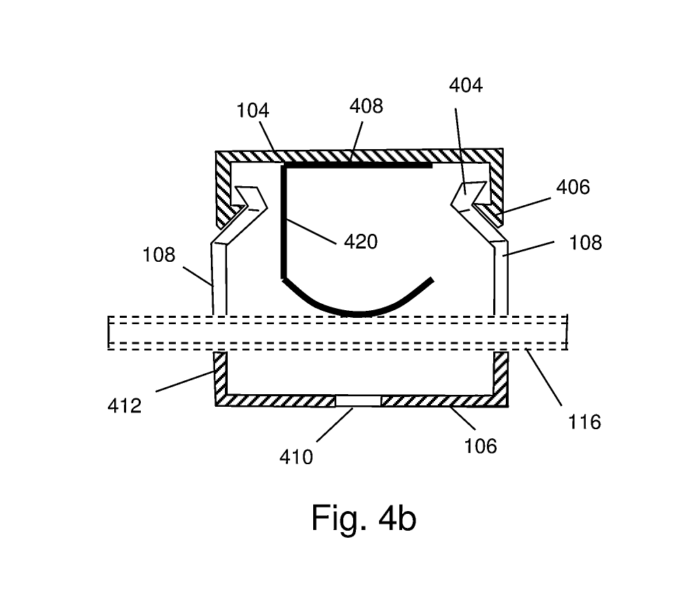

FIG. 4a shows a cross section of the exemplary organizer of FIG. 1 taken through the width of the organizer at a slot position. FIG. 4a shows the cover 104 and the base 102, the base comprising the bottom 106 and sides 412, sides comprising tabs 108. A tube 116 is shown extending through the structure. The tube rests on a turned up edge 416 of the sidewall 412 structure and is further captured by the pressure of the resilient structure 418 and 414 pressing down from the top.

The resilient structure as shown comprises a foam core 418 and an optional skin 414 or shell surrounding the core. The optional skin 414 is preferably non-porous, waterproof covering to allow the organizer to be sterilized by liquid solutions without becoming waterlogged or requiring excessive drying time. Also to improve the holding of the lines, the skin or the foam may have a high friction, or high tack, or mildly adhesive property in relation to the lines, which may be typically vinyl tubing. The foam 110 may be affixed to the cover with adhesive or by other techniques as desired. Open cell foam or foam without the skin that is not easily disinfected may be useful in a one-time use, throw away protocol.

FIG. 4a also shows greater detail in the catch or snap structures for releasably holding the cover to the base. Each tab 108 includes a beveled notch structure 404 at the top for receiving a cover tab 406 on the side of the cover. The beveled notch 404 has an oblique angle at the top side to allow the cover to be removed by pulling upward. Upon pulling the cover upward, the tab springs back allowing the cover to be removed. The cover has an oblique angle on the cover tab 406 to allow installation of the cover by pushing the cover onto the base tabs 108. Upon pressing the cover 104 down onto the base tabs 108 the oblique angle of the cover tab 406 pushes the base tabs 108 back allowing the cover to drop onto the base tabs 108. The lower side of the notch 404 on the tabs has an oblique angle 405 matching the angle of the cover tab 406 for secure resting of the cover 104 on the base 102.

FIG. 4a also shows an alternative foam profile including a chamfered corner 407 at the bottom. Other profiles including rounded or curved profiles may also be used. The chamfered profile may be useful to ease the attachment of the cover to the base. The cover may be attached by pushing the cover down onto the base. Alternatively, the cover may be tilted sideways and one side may be placed in the notch 404. Then, the cover may be tilted and slipped into the side tab notches on a first side. Then using the first side tab notches as a hinge, rotating the cover to level position where the second cover side engages the second set of base tabs. A slight push against the hinge notches will further ease the engagement of the second side.

FIG. 4a also indicates a position for a mounting hole. A keyhole slot 410 or other mounting structure may allow easy mounting to a bed rail or other support. The bed rail should have corresponding screws or posts to accommodate the keyhole notches.

In a further alternative, a directional cover may be implemented by asymmetrical latch features. For example the right and left latch features of FIG. 4a may be different sizes, such that a smaller latch feature 404a, 406a (shown same size in FIG. 4a) on the left will not work with a larger latch feature 404, 406 on the right. Thus, the cover would only fit one way and not reversed.

FIG. 4b illustrates an exemplary alternative resilient structure. FIG. 4b illustrates a spring 420 for pressing on the tube 116 when the cover 104 is in place. The spring may be attached to the channel by adhesive 408 or by other attachments, for example but not limited to screws, rivets, plastic welding, or other techniques. The spring may be plastic, steel, stainless steel, coated steel, or other material suitable for fabricating springs. Other spring shapes may be used. A leaf spring is shown, a coil spring may be used. The spring may preferably be one spring per slot for independent action on each tube.

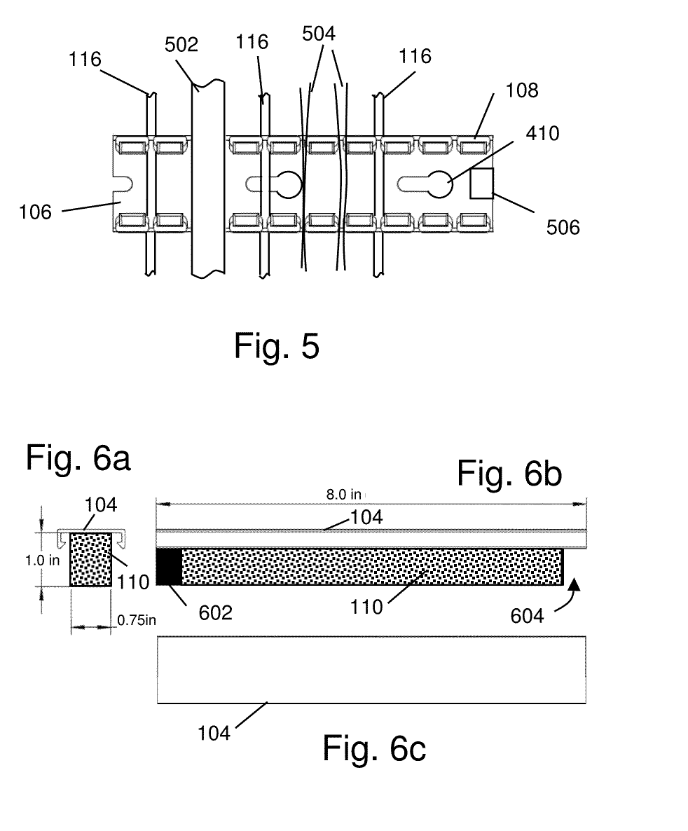

FIG. 5 shows a top view of the exemplary organizer base section loaded with various tubes and wires. FIG. 5 shows a combination of lines including a large tube 502 and small wires 504 loaded along with more typical sized tubes 116. The position of the large tube also shows the removal of base tabs 108 to allow passage of the larger tube 502. The small wires 504 may be loaded with several wires per slot, if desired. FIG. 5 also shows the keyhole slot 410 for mounting the base on a bed rail. Other mounting structures may also be used.

FIG. 6a through FIG. 6c show various views of the exemplary cover concept of the organizer of FIG. 1. FIG. 6a shows an end view of the exemplary cover 104 assembly showing the cover structure 104 with attachment tabs along with a foam resilient structure 110. The foam resilient structure 110 has a rectangular cross section and may be attached to the cover structure with adhesive. Exemplary dimensions are shown. Other dimensions may be used as desired.

FIG. 6b shows a side view of the cover 104 showing the cover sides and foam 110. The length dimension should preferably match the length dimension of the base. The cover may be formed by extrusion due to the constant profile. In one variation, the cover may be supplied with scoring or perforations to allow breaking of the cover to shorter lengths, if desired. Thus, the length may be determined at the point of use without requiring special tools.

FIG. 6c shows a top view of the cover. The top is smooth and flat and may be used for labeling the lines.

In one alternative, the cover may be divided into multiple covers placed end to end. For example the cover may be divided in half allowing half of the slots to be loaded an secured before loading the other half. Alternatively, one half may be removed while retaining the other half undisturbed. For example, dialysis lines may be removed while retaining IV and oxygen lines undisturbed. The organizer may be supplied with the cover divided, or the cover may be divided, as desired, at the point of use.

In further alternatives, the organizer may include a feature to prevent reverse installation of the cover. Without writing on the cover, the cover may be installed left to right or right to left. Both ways are equivalent and indistinguishable. If however, lines are labeled on the cover, the labels would be mismatched if the cover were removed and reinstalled in the reverse direction (rotated 180 degrees). Thus, one variation may add a feature to the cover having a complementary feature in the base that prevents installation of the cover in the reverse direction. For example, the base may have a post (FIG. 5, ref 502) off center to one side that corresponds to a hole 604 in the foam. If the cover were attempted to be installed in reverse direction, the post and hole would not line up and installation would be prevented. To further strengthen the mismatch, a solid post 602 may be placed on the cover to interfere with the base post 506 if the cover is reversed. See FIG. 6 ref 602.

In a further alternative, the alignment feature may comprise a marking on the cover to indicate which side is toward or away from the patient. For example, an arrow indicating “patient side” may help prevent reversal of the cover.

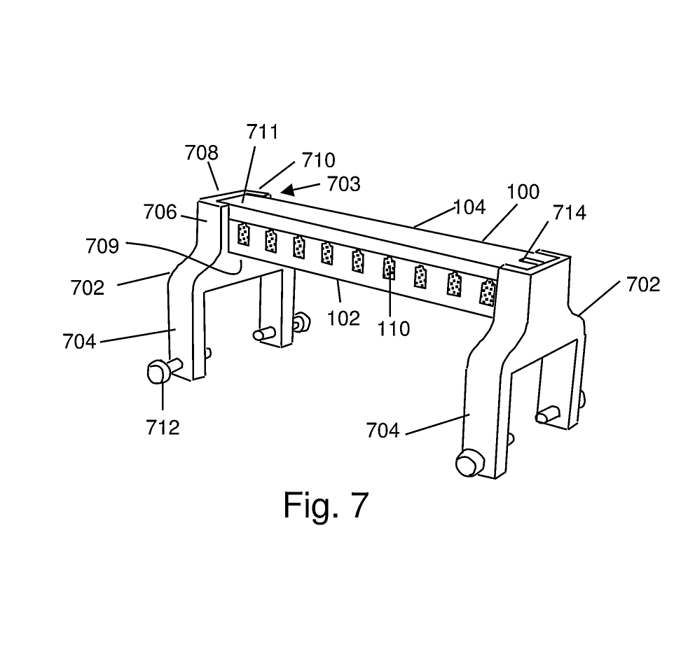

FIG. 7 illustrates an exemplary holder and organizer for attaching to a support such as, for example, a bed rail. The holder 702 forms a channel 703 at the top for receiving one end of the organizer 100. The bottom of the holder 702 comprises a clamp comprising two arms 704 with adjusting screws 712 for clamping to opposite sides of a bed rail or other structure. The top channel includes support on four sides, front 706, back 708, bottom 709, and end 710. The top 711 is open to receive the organizer 100. Two holders may be placed on the bed rail and the clamp screws adjusted for the desired holding force. The organizer may be then placed in the slots between the two holders. The top of the organizer may be removed while leaving the base in the holders. The organizer may be held in the holders by gravity and a slight amount of friction. The holders may be placed any distance apart to accommodate the desired length of organizer.

The open top of the holder allows for quick and easy mobility of the patient. If the patient must be moved from the bed or to another bed, the organizer may be simply lifted out of the holder with the lines remaining in the organizer. The organizer may then be kept with the patient and the lines and moved to another location.

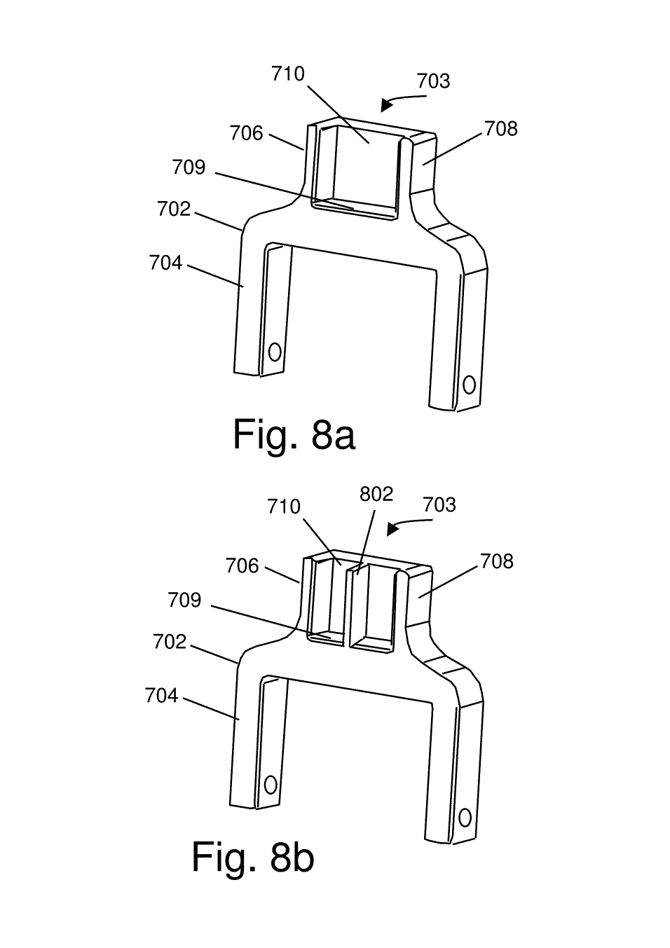

A keyway notch 714 may be provided in the cover 104 for use with a keyed holder (see FIG. 8b) to provide positive orientation of the cover and prevent reversal of the cover.

FIG. 8a and FIG. 8b illustrate exemplary holders for supporting the organizer and attaching the organizer to a bed rail or other support. FIG. 8a shows a universal holder fitting several organizer ends. FIG. 8b shows an exemplary keyed holder fitting only correspondingly keyed organizer ends.

The key of FIG. 8b comprises a vertical rib 802 within the channel 703 for the organizer end. Assembling the organizer into the channel 703 of the holder requires that the organizer have a corresponding key slot cut into the base 102 and the cover 104. Thus, the organizer is fabricated with one end having the corresponding key slots and the other end fully intact. One bracket having a key rib 802 is attached to the bed rail and a second holder without the key rib 802 is assembled opposite the first bracket. Thus, the organizer, and in particular, the cover can fit between the two holders in only one direction. The key is shown centered; however, the key may be off center. Multiple ends with multiple different key positions allow for matching a number of different organizers, if desired.

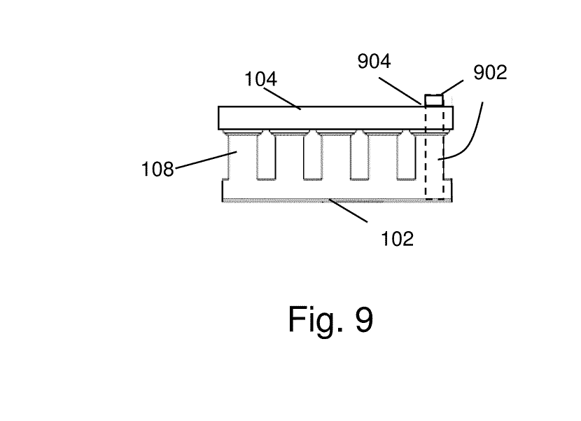

FIG. 9 illustrates a side view of an alternative exemplary cover key comprising a post in the base extending through a hole in the cover. Referring to FIG. 9, shows an exemplary organizer having a length of five tabs 108 and including four slots between the tabs. A cover 104 is shown assembled onto the base at the top of the tabs. An exemplary key is shown comprising a post 902 mounted on the base and extending up through a hole 904 in the cover. The opposite end of the cover has no hole. Thus, the cover cannot be assembled onto the base with the cover reversed in direction because of the interference of the post with the cover. The post 902 may be any desired size or cross section shape—round, square, rectangular, hexagonal or other shape. The length should be sufficient to prevent reversed assembly of the cover on the base. The post may be centered across the width of the base, however, off center may also work. Alternatively, the post may be attached to the cover and the hole may be in the base.

### CONCLUSION

Relative terms such as “bottom” and “top” with respect to features shown in the drawings typically refer to the orientation of drawing features relative to the page and are for convenience of explanation only. The device itself may be operated in any orientation relative to gravity. In this disclosure, typical exemplary ranges may be provided. It is intended that ranges given include any sub-range within the provided range.

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

What is claimed is:

1. A device for organizing medical lines, said medical lines including tubing and/or wires, said device comprising:
a base structure and a cover structure;
said base structure forming a channel structure comprising a bottom member and two sides attached to said bottom member, said two sides comprising a first side and a second side opposite one another across from said bottom member;
each side of said two sides comprising one or more tabs forming one or more slots between said one or more tabs, said one or more slots for receiving said medical lines;
said one or more tabs comprising a base latch structure for receiving and holding said cover structure;
said cover structure for forming a bridge between said first side and said second side, said cover structure having a cover latch structure for interfacing with said tab latch structure of said first side and said tab latch structure of said second side for attaching said cover structure to said base structure;
said one or more tabs forming said base latch structure being flexible to releasably engage said cover latch structure of said cover structure;
said cover structure comprising a resilient restraint structure for pressing against at least one line of said medical lines for capturing and restraining said at least one line within a slot of said one or more slots.

2. The device in accordance with claim 1, wherein said one or more tabs have a wider top compared with a tab width at center height to capture said medical lines when said cover is detached.

3. The device in accordance with claim 2, wherein said tabs are sufficiently flexible to allow entry of medical lines wider than an entry opening between said tabs.

4. The device in accordance with claim 3, wherein at least one of said one or more tabs is configured to break away or bend away sufficiently to allow joining of two adjacent slots to allow a wider tube than allowed by one slot of said two adjacent slots.

5. The device in accordance with claim 1, wherein said resilient restraint structure comprises foam.

6. The device in accordance with claim 5, wherein said foam has a resilient non-porous skin to prevent entry of liquid and facilitate disinfection of said device.

7. The device in accordance with claim 1, wherein said resilient restraint structure comprises a spring structure.

8. The device in accordance with claim 1, wherein said cover structure has a flat surface allowing labeling of each said tube or other line.

9. The device in accordance with claim 1, wherein said base structure further forming a keyhole slot for mounting said device on a support structure.

10. The device in accordance with claim 1, wherein said device further comprises at a first holder for receiving and holding said cover structure and base structure; said first holder comprising a clamp structure for attaching to a support structure; said first holder forming a cavity for receiving one end of said cover structure and said base structure.

11. The device in accordance with claim 10, wherein said device further comprises a second holder at an opposite end of said base structure from said first holder, said second holder having sides restraining three sides and one end of said assembly; said first holder and said second holder allowing insertion and removal of said assembly from a top direction and providing restraint in lateral and downward directions.

12. The device in accordance with claim 11, wherein said second holder comprises a key rib and said cover forms a key slot in one end to align with said key rib of said second holder to prevent installation of said cover in a reversed direction.

13. The device in accordance with claim 1, further including a feature on said cover structure and a complementary feature on said base structure arranged to interfere if said cover structure is attempted to be installed in reverse.

14. The device in accordance with claim 13, wherein said complementary feature on said base structure is a post positioned off of the longitudinal center of said device and said feature on said cover structure is a post positioned opposite the longitudinal center of said device from said post on said base structure; said post on said base structure and said post on said cover structure having sufficient total length to interfere and prevent installing of the cover structure on the base structure when the cover structure is attempted to be installed in reverse.

15. A method for organizing medical lines, said medical lines including tubing and/or wires, said method comprising:
providing a base structure and a cover structure;
said base structure forming a channel structure comprising a bottom member and two sides attached to said bottom member, said two sides comprising a first side and a second side opposite one another across from said bottom member;
each side of said two sides comprising one or more tabs forming one or more slots between said one or more tabs, said one or more slots for receiving said medical lines;
said one or more tabs comprising a base latch structure for receiving and holding said cover structure;
said cover structure for forming a bridge between said first side and said second side, said cover structure having a cover latch structure for interfacing with said tab latch structure of said first side and said tab latch structure of said second side for attaching said cover structure to said base structure;
said one or more tabs forming said base latch structure being flexible to releasably engage said cover latch structure of said cover structure;
said cover structure comprising a resilient restraint structure for pressing against at least one line of said medical lines for capturing and restraining said at least one line within a slot of said one or more slots;
placing at least one line of said medical lines through a first slot on said first side and through a second slot on said second side, said second slot opposite said first slot;
installing said cover on said base by engaging said latch structures on said first side and said second side; and
pressing said resilient structure on said at least one line to restrain said at least one line from moving longitudinally.

16. The method in accordance with claim 15, wherein the step of installing said cover comprises:
engaging said first latch on said first side without engaging said second latch on said second side;
rotating said cover with said first latch as a pivot;
engaging said second latch while maintaining said engagement of said first latch.

17. The method in accordance with claim 15, further including:
removing a first tab to create a slot larger than said first slot.

18. The method in accordance with claim 15, wherein said resilient structure comprises foam.

19. The method in accordance with claim 18, wherein said foam has a non-porous skin.

20. The method in accordance with claim 15, wherein said resilient structure comprises a spring.
